(19) United States
(12) Patent Application Publication
(10) Pub. No.: US 2026/0115054 A1
(43) Pub. Date: Apr. 30, 2026

(54) SEMICONDUCTOR GAS SENSOR, DIAPER, METHOD FOR DETECTING GAS, METHOD FOR DETECTING URINE AND/OR FECES, METHOD FOR DETERMINING DIAPER REPLACEMENT TIME, AND GAS-RESPONSIVE MATERIAL

(71) Applicant: TOHOKU UNIVERSITY, Sendai-shi (JP)

(72) Inventors: Shu Yin, Sendai-shi (JP); Lei Miao, Sendai-shi (JP); Yibei Xue, Sendai-shi (JP); Ayahisa Okawa, Sendai-shi (JP); Takuya Hasegawa, Sendai-shi (JP)

(73) Assignee: TOHOKU UNIVERSITY, Sendai-shi (JP)

(21) Appl. No.: 19/370,025

(22) Filed: Oct. 27, 2025

(30) Foreign Application Priority Data
Oct. 31, 2024 (JP) 2024-191987

(51) Int. Cl. A61F 13/42; G01N 27/12; G01N 33/00

(52) U.S. Cl. CPC A61F 13/42; G01N 27/12; G01N 33/0027; A61F 2013/424

(57) ABSTRACT

An object of the invention is to provide a semiconductor type gas sensor capable of distinguishing and detecting an ammonia gas and a hydrogen sulfide gas with high sensitivity without heating to a high temperature (about 180° C. to 300° C.), a method for detecting gases using the semiconductor type gas sensor, a diaper, a method for detecting urine and/or feces, a method for determining a diaper replacement time, and a gas-sensing material used in the semiconductor type gas sensor. To provide the semiconductor type gas sensor, which includes an electrode and a VO₂(M1) phase containing layer provided on the electrode and in which a VO₂(M1) phase functions as the gas-sensing material, the diaper, the method for detecting gases, the method for detecting urine and/or feces, the method for determining a diaper replacement time, and the gas-sensing material.

### CROSS REFERENCE TO RELATED APPLICATIONS

This application claims priority based on Japanese Patent Application No. 2024-191987 filed on Oct. 31, 2024, the content of which is incorporated herein by reference in its entirety.

### BACKGROUND OF THE INVENTION

### 1. Field of the Invention

The present invention relates to a semiconductor type gas sensor, a diaper, a method for detecting gases, a method for detecting urine and/or feces, a method for determining a diaper replacement time, and a gas-sensing material.

### 2. Description of Related Art

A gas sensor is used as a sensor for detecting presence or absence of a gas and a concentration thereof.

As one gas sensor, a semiconductor type gas sensor (also referred to as a “chemical resistance gas sensor”) using a change in electrical resistance accompanied by a reaction between a metal oxide semiconductor which is a gas-sensing material and a gas is known. Semiconductor type gas sensors are used in various applications, for example, alarms for various harmful gases, portable sensors for detecting gas types in exhaled breath, odor sensors, and monitoring working environments, due to advantages such as excellent mass productivity and relatively inexpensive manufacturing.

In a semiconductor type gas sensor using an n-type semiconductor, electrical resistance decreases when exposed to a reducing gas (electron donating gas), and the electrical resistance increases when exposed to an oxidizing gas (electron withdrawing gas). Conversely, in a semiconductor type gas sensor using a p-type semiconductor, electrical resistance increases when exposed to a reducing gas (electron donating gas), and the electrical resistance decreases when exposed to an oxidizing gas (electron withdrawing gas). Thus, in the semiconductor type gas sensor, a response (change in electrical resistance) is determined according to a type (n-type or p-type) of the metal oxide semiconductor which is a gas-sensing material and a gas type to be detected (reducing or oxidizing). Therefore, a technique for selectively detecting a specific gas using the semiconductor type gas sensor has been developed.

For example, PTL 1 discloses a gas sensor capable of reducing an influence of a combustible gas such as hydrogen and an influence of an oxidizing gas such as NOₓ to improve detection sensitivity (gas sensitivity) and a response to an odorous gas as compared with the related art. The gas sensor includes a gas detection layer containing SnO₂ in a content of 90% by mass or more and Ir, P, and Pt in a specific content.

PTL 2 discloses an ammonia gas sensor having high ammonia selectivity and improved measurement accuracy. The gas sensor includes a first solid electrolyte layer containing one or more oxides (ammonia-selective oxides) selected from the group consisting of V, Bi, and Sb as a main component, and a reference electrode and a detection electrode formed on a surface of the first solid electrolyte layer.

PTL 3 discloses a semiconductor type gas sensor in which a new gas detection principle (a change in resistance switching characteristics at an electrode interface due to a gas) is combined with an operation principle of the semiconductor type gas sensor (an oxidation-reduction reaction on a surface of the gas sensor caused by gas surface adsorption), thereby enabling identification of a hydrogen gas while maintaining a highly sensitive detection capability of the semiconductor type gas sensor for a wide range of gas types.

In general, the semiconductor type gas sensor does not easily react with a gas at room temperature, and in order to function as a desired high-sensitivity gas sensor, it is necessary to heat the gas detection layer to a high temperature (about 180° C. to 300° C.) for activation. However, this high temperature heating has been pointed out to have problems such as making it difficult to reduce a size of a device and causing peeling between the gas detection layer and a substrate. In order to solve this problem, for example, as disclosed in PTLs 4 and 5, it has been proposed to provide an adhesion layer for preventing peeling between a gas detection layer and a substrate.

A technique for detecting a use state of a human care product such as a diaper with various sensors has been developed.

For example, PTL 6 discloses a diaper replacement time detection sensor including a pair of electrodes for detecting urination, and discloses that a wet state of the diaper due to urination can be grasped by measuring electrical characteristics (for example, a resistance value or conductivity) between the electrodes by the diaper replacement time detection sensor.

PTL 7 discloses an excretion detection device separately including a temperature detection unit that detects a temperature inside a diaper and a detection unit that detects a state of diaper soiling.

PTL 8 discloses use of a gas sensor including a heater and an element portion made of a sintered body of SnO₂ as a metal oxide semiconductor as a unit for detecting defecation, in addition to a moisture sensor for detecting urination, and discloses that the gas sensor can detect methyl mercaptan (methanethiol), which is a main component of odor of defecation, with high sensitivity.

### Citation List

### Patent Literature

PTL 1: WO2009/130884

PTL 2: JP2010-139238A

PTL 3: JP2022-179881A

PTL 4: JP2006-317155A

PTL 5: WO2009/078370

PTL 6: JPH09-033468A

PTL 7: JPH09-290001A

PTL 8: JP2022-058409A

### Non Patent Literature

NPL 1: J.X. Wang, X.W. Sun, et al. Nanotechnology, 2009, Vol. 20, p.465501.

NPL 2: M. Sinha, S. Neogi, et al. Sens. Actuators, B Chem. 2021, Vol. 336, p.129729.

### SUMMARY OF THE INVENTION

The present inventors have come up with the idea that if a technique capable of detecting urine and feces by the same sensor can be achieved by using a semiconductor type gas sensor, it is possible to develop a diaper product having an added value that has not been found in the related art. However, the semiconductor type gas sensor that selectively responds by distinguishing an ammonia gas derived from urine and a hydrogen sulfide gas derived from feces has not been known. Further, as described above, the semiconductor type gas sensor requires high temperature heating for activation of the gas detection layer in gas sensing, and in a diaper product used at body temperature, it is a high hurdle to create the semiconductor type gas sensor that distinguishes and detects urine and feces.

The invention has been made in view of the above various circumstances, and an object of the invention is to provide a semiconductor type gas sensor capable of distinguishing and detecting an ammonia gas and a hydrogen sulfide gas with high sensitivity without heating to a high temperature (about 180° C. to 300° C.), and a method for detecting gases using the semiconductor type gas sensor.

An object of the invention is to provide a diaper, a method for detecting urine and/or feces, and a method for determining a diaper replacement time, which enable detection of urine and feces without heating to a high temperature (about 180° C. to 300° C.) by using the semiconductor type gas sensor according to the invention.

An object of the invention is to provide a gas-sensing material capable of distinguishing and detecting the ammonia gas and the hydrogen sulfide gas with high sensitivity without heating to a high temperature (about 180° C. to 300° C.).

Among metal oxide semiconductors, those exhibiting a gas response at a relatively low temperature have been reported. For example, NPL 1 discloses that ZnO, which is a typical p-type semiconductor, exhibits a p-type behavior to an NO₂ gas at a temperature of 100° C. or lower, and a sensing behavior is reversed at an operating temperature exceeding 100° C. Further, according to NPL 2, a ZnO/CNT composite can reverse a sensing behavior for each gas by temperature control.

Based on the matters disclosed in NPLs 1 and 2, the present inventors have conceived that a unique semiconductor material having a narrow band gap can also modulate a sensing behavior depending on a temperature, and have repeated studies. As a result, it has become clear that a semiconductor type gas sensor using a VO₂(M1) phase, which is an n-type semiconductor and has a narrow band gap of 0.68 eV, as a gas-sensing material exhibits a characteristic gas response behavior (positive change in electrical resistance) different from that of the semiconductor type gas sensor in the related art with respect to an ammonia gas at a low temperature of 20° C., and can distinguish and detect the ammonia gas and a hydrogen sulfide gas or the like, which exhibits a negative change in electrical resistance, with high sensitivity without being heated to a high temperature (about 180° C. to 300° C.). Based on these findings, the invention has been completed through further studies.

The above problems of the invention are solved by the following units.

### 1

A semiconductor type gas sensor includes: an electrode; and a VO₂(M1) phase containing layer provided on the electrode, in which a VO₂(M1) phase functions as a gas-sensing material.

### 2

In the semiconductor type gas sensor according to <1>, the electrode and the VO₂(M1) phase containing layer are provided on/above a substrate in this order.

### 3

The semiconductor type gas sensor according to <1> or <2> is for use in distinguishing and detecting an ammonia gas and at least one gas among hydrogen sulfide, hydrogen, nitrogen monoxide, toluene, acetone, and ethanol.

### 4

The semiconductor type gas sensor according to any one of <1> to <3> is for use at an operation temperature of 15° C. to 65° C.

### 5

The semiconductor type gas sensor according to any one of <1> to <4> has an increase in electrical resistance when exposed to an ammonia gas, and has a decrease in electrical resistance when exposed to at least one gas of hydrogen sulfide, hydrogen, nitrogen monoxide, toluene, acetone, and ethanol.

### 6

In the semiconductor type gas sensor according to <2>, the substrate is a flexible substrate.

### 7

The semiconductor type gas sensor according to <5> is for use in detection of urine and/or feces.

### 8

The semiconductor type gas sensor according to <7> is for use by being attached to a diaper.

### 9

A diaper includes: the semiconductor type gas sensor according to <8>.

### 10

A method for detecting gases includes: distinguishing and detecting an ammonia gas and at least one gas among hydrogen sulfide, hydrogen, nitrogen monoxide, toluene, acetone, and ethanol using the semiconductor type gas sensor according to any one of <1> to <8>.

### 11

A method for detecting urine and/or feces includes: distinguishing and detecting urine and feces using the semiconductor type gas sensor according to any one of <1> to <8>.

### 12

A method for determining a diaper replacement time includes: determining a diaper replacement time by detecting an ammonia gas derived from urine and/or a hydrogen sulfide gas derived from feces excreted on a diaper using the semiconductor type gas sensor according to <7>.

### 13

A gas-sensing material contains a VO₂(M1) phase as an active component.

### 14

The gas-sensing material according to <13> is for use in distinguishing and detecting an ammonia gas and at least one gas among hydrogen sulfide, hydrogen, nitrogen monoxide, toluene, acetone, and ethanol.

### 15

The gas-sensing material according to <14> is for use in detection of urine and/or feces.

In the invention, a numerical range expressed with “to” means a range that includes numerical values written before and after “to” as a lower limit value and an upper limit value.

According to the semiconductor type gas sensor in the invention and the method for detecting gases using the semiconductor type gas sensor, an ammonia gas and a hydrogen sulfide gas or the like can be distinguished and detected with high sensitivity without heating to a high temperature (about 180° C. to 300° C.).

According to the diaper, the method for detecting urine and/or feces, and the method for determining a diaper replacement time in the invention, the semiconductor type gas sensor in the invention is provided, and can distinguish and detect urine and feces without heating to a high temperature (about 180° C. to 300° C.).

The gas-sensing material in the invention makes it possible to distinguish and detect the ammonia gas and the hydrogen sulfide gas or the like with high sensitivity without heating to a high temperature (about 180° C. to 300° C.).

### BRIEF DESCRIPTION OF THE DRAWINGS

FIG. 1 illustrates an XRD pattern of a single phase VO₂(M1) powder obtained in Synthesis Example 1 (in a nitrogen gas atmosphere);

FIG. 2 illustrates XRD patterns of the powder obtained in Synthesis Example 1 (in the atmosphere);

FIG. 3 illustrates (a) an XRD pattern of the VO₂(M1) phase (ICDD No. 43-1051 VO₂(M1)) registered in ICDD, and illustrates (b) an XRD pattern of a VO₂(B) phase (ICDD No. 81-2392 VO₂(B)) registered in ICDD;

FIGS. 4A and 4B are photographs substituting for a drawing of a flexible interdigitated electrode sheet produced in Example;

FIG. 5 is a photograph substituting for a semiconductor type gas sensor chip produced in Example;

FIG. 6 is a graph illustrating results of gas responses of the semiconductor type gas sensor using a VO₂(M1) phase single phase layer as a sensing layer to various gas types at 20° C.;

FIG. 7 is a graph illustrating results of gas responses of the semiconductor type gas sensor using the VO₂(M1) phase single phase layer as the sensing layer to the various gas types at 20° C.;

FIG. 8 is a bar graph illustrating results of gas responses of the semiconductor type gas sensor to the various gas types at 20° C., 30° C., and 40° C. using the VO₂(M1) phase single phase layer as the sensing layer;

FIG. 9 is graphs illustrating results of gas responses of the semiconductor type gas sensor using a layer of the mixed phase of the VO₂(M1) phase and the V₆O₁₃ as the sensing layer to the various gas types at 20° C.;

FIG. 10 is a radar chart illustrating results of gas responses of the semiconductor type gas sensor using the layer of the mixed phase of the VO₂(M1) phase and the V₆O₁₃ as the sensing layer to the various gas types at 20° C., 30° C., 40° C., 50° C., and 60° C.;

FIG. 11 is a graph illustrating responses of a diaper sensor (inside mode) to three supplies of 40 mL, 38° C. artificial urine at 25° C.;

FIG. 12 is a graph illustrating responses of the diaper sensor (inside mode) to three supplies of 5 ppm H₂S gas at 25° C.;

FIG. 13 is a graph illustrating responses of the diaper sensor (outside mode) to three supplies of 40 mL, 38° C. artificial urine at 25° C.;

FIG. 14 is a graph illustrating responses of the diaper sensor (outside mode) to three simultaneous supplies of 5 ppm H₂S gas and 40 mL, 38° C. artificial urine at 25° C.

### DESCRIPTION OF EMBODIMENTS

Preferred embodiments of the invention will be described below, but the invention is not limited to the following embodiments except for being specified in the invention.

Semiconductor type gas sensor The semiconductor type gas sensor according to the invention includes: an electrode; and a VO₂(M1) phase containing layer provided on the electrode, in which a VO₂(M1) phase functions as a gas-sensing material.

That is, the semiconductor type gas sensor according to the invention is a gas sensor utilizing the fact that electrical resistance of a gas sensor changes depending on a gas type due to a reaction between the VO₂(M1) phase, which is a metal oxide semiconductor, and the gas.

In the invention, the “VO₂(M1) phase” means VO₂(Monoclinic, P2₁/c), and is an n-type semiconductor that causes a reversible metal-semiconductor transition (MST) with a metal phase VO₂(R) phase at about 68° C. The fact that the VO₂(M1) phase is an n-type semiconductor is described in A. Patel, et al. Superlattices Microst. 2019, Vol. 130, p.160-167., Y. Zhou, et al. J. Appl. Phys. 2013, Vol. 113, p.213703.

In addition to the VO₂(M1) phase, a VO₂(B) phase is known as VO₂, which is a metal oxide semiconductor. This VO₂(B) phase is VO₂(Monoclinic, C₂/m), and transitions to a metal phase VO₂(R) phase at about 480° C.

In the VO₂(B) phase, it is known that electrical resistance of the VO₂(B) phase increases at 20° C. due to contact with any gas of an ammonia gas, a hydrogen sulfide gas, an acetone gas, an ethanol gas, and a toluene gas, which are reducing gases (see V. X. Hien, et al., J. Mater. Sci., 2021, Vol. 32, p.13803-13812.).

In contrast, in the semiconductor type gas sensor according to the invention, for example, when a single phase of a VO₂(M1) phase is used as the gas-sensing material, at least at 20° C. to 40° C., electrical resistance decreases due to contact with the hydrogen sulfide gas, a hydrogen gas, the toluene gas, the acetone gas, and the ethanol gas, which are the reducing gases, and a nitrogen monoxide gas, which is an oxidizing gas, while the electrical resistance increases due to contact with the ammonia gas, which is the reducing gas (see Example 1 (1) described later). When a mixed phase of the VO₂(M1) phase and V₆O₁₃ is used, at least at 20° C. to 60° C., the electrical resistance decreases due to contact with the hydrogen sulfide gas, the hydrogen gas, the toluene gas, the acetone gas, and the ethanol gas, which are the reducing gases, and the nitrogen monoxide gas, which is the oxidizing gas, while the electrical resistance increases due to contact with the ammonia gas, which is the reducing gas (see Example 1 (2) described later). A reason why a temperature range in which responses to gas types is maintained is wider in the mixed phase of the VO₂(M1) phase and the V₆O₁₃ compared with in the single phase of the VO₂(M1) phase is presumed, but it is considered that there is a possibility of being related to the fact that, due to the mixed phase with the V₆O₁₃, the Schottky barrier described later becomes larger than that in a case of the single phase of the VO₂(M1) phase, and it is considered that the similarity may occur in a mixed phase with vanadium oxide other than the V₆O₁₃.

In the semiconductor type gas sensor according to the invention, a reason why the gas response is reversed due to the temperature is considered as follows.

The Schottky junction may be formed at an interface between the electrode that is a metal and the VO₂(M1) phase that is an n-type semiconductor due to contact between the electrode and the VO₂(M1) phase. It is considered that when the semiconductor type gas sensor according to the invention comes into contact with the ammonia gas, an electron affinity (and work function) of the VO₂(M1) phase decreases due to adsorption of the ammonia gas, and the Schottky barrier (value calculated by subtracting [electron affinity of VO₂(M1) phase containing layer] from [work function of electrode]) exceeds 0 (Schottky contact), resulting in an increase in resistance. Meanwhile, in the contact between the semiconductor type gas sensor according to the invention and the hydrogen sulfide gas, the hydrogen gas, the nitrogen monoxide gas, the toluene gas, the acetone gas, or the ethanol gas, the electron affinity (and work function) of a VO₂(M1) phase surface is unlikely to decrease due to gas adsorption, and the Schottky barrier becomes less than 0 (ohmic contact), resulting in a decrease in resistance.

In the following description, “hydrogen sulfide gas or the like” means “at least one gas of the hydrogen sulfide gas, the hydrogen gas, the nitrogen monoxide gas, the toluene gas, the acetone gas, and the ethanol gas”.

That is, in the semiconductor type gas sensor according to the invention, usually, when exposed to the hydrogen sulfide gas or the like, the electron affinity of the VO₂(M1) phase containing layer changes, and the Schottky barrier of the Schottky junction formed between the VO₂(M1) phase containing layer and the electrode becomes less than 0 (ohmic contact). Meanwhile, when exposed to the ammonia gas, different changes in the electron affinity of the VO₂(M1) phase containing layer occur, and the Schottky barrier exceeds 0 (Schottky contact).

As a result, the electrical resistance of the semiconductor type gas sensor according to the invention decreases when exposed to the hydrogen sulfide gas or the like, and the electrical resistance increases when exposed to the ammonia gas.

As described above, the semiconductor type gas sensor according to the invention can be used as a semiconductor type gas sensor for distinguishing and detecting the hydrogen sulfide gas or the like and the ammonia gas.

### Gas-Sensing Material

The VO₂(M1) phase containing layer is not particularly limited as long as the VO₂(M1) phase containing layer is a layer containing the VO₂(M1) phase and functioning as the gas-sensing material. For example, the VO₂(M1) phase containing layer may be a VO₂(M1) phase single phase layer or a layer containing the VO₂(M1) phase and components other than the VO₂(M1) phase. Examples of the components other than the VO₂(M1) phase include vanadium oxide other than the VO₂(M1) phase (referred to as “other vanadium oxide”).

The other vanadium oxide may be a compound having a crystal structure such as a monoclinic phase or a tetragonal phase and containing a vanadium element or a composite compound thereof. Examples of the other vanadium oxide include vanadium oxide in which V₆O₁₃, V₂O₅, V₄O₉, VO, a VO₂(B) phase, a VO₂(A) phase, or NH₄VO₃, or any of these doped with a cation such as W or Mo, or an anion such as N, B, or P.

A content of the VO₂(M1) phase in the VO₂(M1) phase containing layer is not particularly limited as long as the VO₂(M1) phase functions as a gas-sensing material and effects of the invention are exhibited. For example, as for a molar ratio, VO₂(M1) phase: the other vanadium oxide=1:99 to 100:0 is preferable, and VO₂(M1) phase: the other vanadium oxide=35:65 to 100:0 is more preferable. As for a layer of the mixed phase of the VO₂(M1) phase and the other vanadium oxide, VO₂(M1) phase: the other vanadium oxide=40:60 to 80:20 is further more pr₂ferable. VO₂(M1) phase: the other vanadium oxide=100:0 means that th₂ VO₂(M1) phase containing layer is a VO₂(M1) phase single phase layer.

The content of the VO₂(M1) phase in the VO₂(M1) phase containing layer is a value obtained by performing powder X-ray diffraction (XRD) on the VO₂(M1) phase containing layer and calculating a ratio of peak areas having the strongest intensities of each phase.

In the semiconductor type gas sensor according to the invention, the VO₂(M1) phase functions as the gas-sensing material, is brought into contact with a gas to be detected, and detects presence or absence of a specific gas type in the gas to be detected based on a gas response pattern of the semiconductor type gas sensor. In the invention, “detect” is not limited to determining the presence or absence of the specific gas type, and includes predicting (estimating) the presence or absence of the specific gas type with desired accuracy. In the invention, “presence” in the “presence or absence” of a specific gas type may mean that a specific gas type is simply present, or may mean that a specific gas type is present in a certain amount (a certain concentration) or more, rather than merely being present. Similarly, “absence” may mean that there is no specific gas type at all (equal to or less than a detection limit), or may mean that an amount (concentration) of the specific gas type is equal to or less than a certain amount (certain concentration) even if the specific gas type is present. Thus, a criterion of the “presence or absence” is appropriately set according to a purpose.

In the semiconductor type gas sensor according to the invention, a response pattern to the hydrogen sulfide gas or the like and a response pattern to the ammonia gas do not change at least at 15° C. to 65° C., which is a temperature range near a human body temperature (at least at 15° C. to 45° C. in a mixed phase of the (VO₂(M1) phase and the other vanadium oxide). Therefore, it can be suitably used as a sensor for human health care such as a diaper.

The fact that the response pattern does not change means that a sensing behavior when the semiconductor type gas sensor according to the invention is exposed to the gas, that is, whether a behavior of a response ΔR/R (Response (%)) represented by the following formula (A) is upward or downward, does not change. Specifically, the response pattern to the hydrogen sulfide gas or the like always exhibits a downward behavior (n-type behavior: decrease in electrical resistance) in the above temperature range, and the response pattern to the ammonia gas always exhibits an upward behavior (p-type behavior: increase in electrical resistance) in the above temperature range.

The semiconductor type gas sensor according to the invention can be used at an operation temperature of, for example, 15° C. to 45° C. (for example, 15° C. to 65° C. in a mixed phase of the (VO₂(M1) phase and other vanadium oxide).

In the semiconductor type gas sensor according to the invention, a detectable concentration of the specific gas types in the gas to be detected varies depending on the gas types, an environment in which the gas is used, and the like, and for example, the ammonia gas can be detected in a concentration range of 2.5 ppm to 18000 ppm, and the hydrogen sulfide gas or the like can be detected in a concentration range of 0.1 ppm to 10000 ppm.

Contact between the semiconductor type gas sensor according to the invention and the gas to be detected can be performed by the gas to be detected reaching the semiconductor type gas sensor by natural diffusion.

The semiconductor type gas sensor according to the invention usually has a configuration in which the VO₂(M1) phase containing layer is provided on an electrode in contact with the electrode. Other configurations are not particularly limited, and a configuration normally used in a semiconductor type gas sensor can also be adopted in the semiconductor type gas sensor according to the invention.

### Electrode

As the electrode, an electrode usable for a semiconductor type gas sensor can be used without particular limitation. The electrode is preferably an electrode made of a metal having a work function of 3.20 eV to 5.44 eV (preferably 4.05 eV to 4.80 eV) from the viewpoint of further actualizing the gas response depending on positive or negative of the Schottky barriers. Specific preferred examples thereof include silver, molybdenum, iron, platinum, and gold.

The semiconductor type gas sensor according to the invention preferably has a layer configuration having the electrode and the VO₂(M1) phase containing layer in this order on/above a substrate.

As the substrate, a substrate usually used in a semiconductor type gas sensor can be used without particular limitation.

In particular, it is more preferable that the substrate is a flexible substrate, that is, the electrode and the VO₂(M1) phase containing layer are provided on/above the flexible substrate in this order.

A material constituting the flexible substrate is not particularly limited as long as the material is an insulating material, and is preferably water-soluble or biodegradable. Examples thereof include cellulose derivatives such as hydroxypropyl methylcellulose (HPMC), polyester, and polyamide.

When the substrate is a flexible substrate, the semiconductor type gas sensor according to the invention can be used as a flexible small-size semiconductor type gas sensor. For example, it can be suitably used as a semiconductor type gas sensor to be attached to a diaper and used for detecting urine and/or feces.

A size of a semiconductor type gas sensor chip according to the invention is not particularly limited. When used as a small-size semiconductor type gas sensor, for example, a sensor having a size of 10 mm to 50 mm in length, 5 mm to 100 mm in width, and 1μm to 10μm in thickness can be used.

### Gas-Sensing Material

The gas-sensing material according to the invention contains the VO₂(M1) phase as an active component.

The VO₂(M1) phase is as described for the VO₂(M1) phase in the semiconductor type gas sensor according to the invention described above. The VO₂(M1) phase may be a VO₂(M1) phase single phase, or may be a mixed phase of the VO₂(M1) phase and the other vanadium oxide in the semiconductor type gas sensor according to the invention described above.

The gas-sensing material according to the invention can be used as a gas-sensing material for distinguishing and detecting an ammonia gas and a hydrogen sulfide gas or the like. That is, when the gas-sensing material according to the invention is provided on the electrode and used, a response pattern to the hydrogen sulfide gas or the like exhibits a downward behavior (n-type behavior: decrease in electrical resistance), and a response pattern to the ammonia gas exhibits an upward behavior (p-type behavior: increase in electrical resistance). Therefore, the gas-sensing material can be used as a material exhibiting a selective gas response.

The gas-sensing material according to the invention can be used as a gas-sensing material for detection of urine and/or feces.

It is known that about 1.7% to 1.8% urea contained in urine is decomposed into ammonia by bacteria. A volatile gas derived from feces contains about 1% hydrogen sulfide gas. Therefore, by using the gas-sensing material according to the invention capable of distinguishing and detecting the ammonia gas and the hydrogen sulfide gas, urine and feces can be distinguished and detected.

Advantages of the semiconductor type gas sensor according to the invention include the following (1) to (5).
(1) Urine and feces can be distinguished and easily detected without requiring complicated analysis, and a state of the diaper can be determined by a response pattern (n-type behavior and p-type behavior) opposite to that of the ammonia gas and the hydrogen sulfide gas.
(2) The semiconductor type gas sensor is low cost and can be reused.
(3) The semiconductor type gas sensor can operate without being heated to a high temperature, and can be free from a heating device. Therefore, a temperature control heater required for the semiconductor type gas sensor in the related art can be omitted. Since the temperature control heater is not required, the semiconductor type gas sensor can be miniaturized, and an organic substrate having low heat resistance can be used. As a result, a small-size semiconductor type gas sensor using a flexible substrate can be obtained.
(4) Since a sensor chip is excellent in flexibility and is excellent in body compatibility and fitting properties, when the sensor chip is used by being attached to a diaper, a foreign body sensation can be significantly reduced.
(5) Since a water-soluble substrate can also be used, it is easily decomposed and environmentally friendly.

Application of the semiconductor type gas sensor according to the invention is not particularly limited, and the semiconductor type gas sensor can be used in applications in which a gas sensor has been used so far. In view of the above (1) to (5), the semiconductor type gas sensor can be suitably used as a semiconductor type gas sensor in a human care device such as a diaper. As a specific example, urine and feces can be independently detected by providing the semiconductor type gas sensor according to the invention in a diaper worn by people requiring care, young children, or the like who have difficulty in communicating through speech. Further, collective monitoring by a nurse station, alerts for different types of excrement based on different music, an alert by a smartphone in the future, and the like are also assumed.

### Method For Producing Semiconductor Type Gas Sensor

The semiconductor type gas sensor chip according to the invention is not particularly limited, and can be obtained by a usual method known as a method for producing a semiconductor type gas sensor using a metal oxide semiconductor.

For example, the VO₂(M1) phase is added to a solvent such as ethanol and dispersed by ultrasonic waves to prepare a slurry, and the slurry is then dropped over the interdigitated electrode using a pipette or the like, the dropped slurry is dried in a drying oven at 30° C. to 100° C., and the solvent is removed to obtain a semiconductor type gas sensor.

### Method for Producing Gas-Sensing Material

The VO₂(M1) phase can be produced, for example, by the following method.

First, a precursor is prepared by mixing a reducing agent such as hydrazine into a mixed solution of aqueous hydrogen peroxide and V₂O₅. VO₂(M1) phase can be obtained by dispersing the obtained precursor in a solvent such as water and performing a chemical reaction in a subcritical fluid and/or a supercritical fluid (preferably in subcritical water and/or supercritical water).

The VO₂(M1) phase single phase can be obtained by performing the chemical reaction in the subcritical fluid and/or the supercritical fluid in an inert gas atmosphere such as argon gas or nitrogen gas.

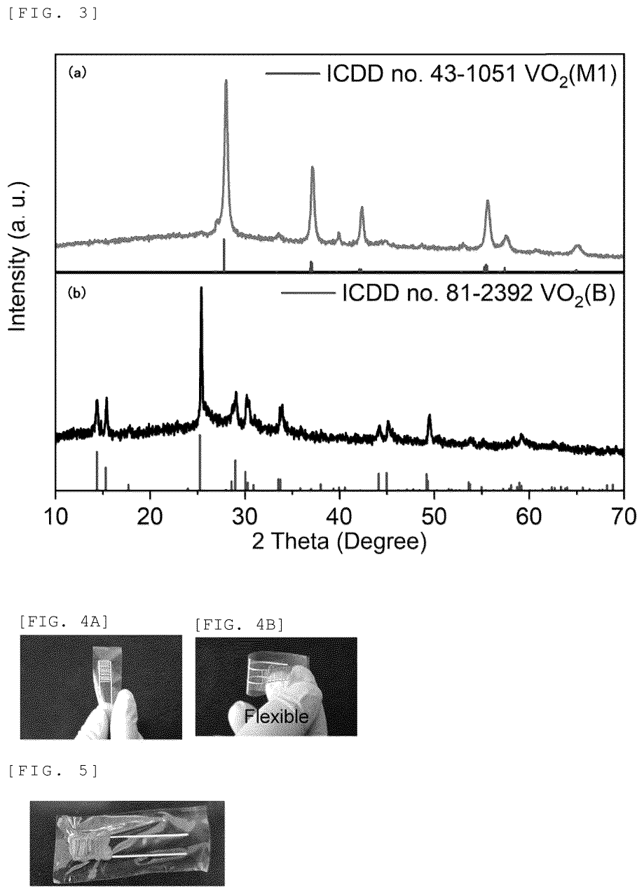

The mixed phase of the VO₂(M1) phase and other vanadium oxide can be obtained by performing a chemical reaction in the subcritical fluid and/or the supercritical fluid in a gas atmosphere containing an oxygen gas (for example, in the atmosphere). A proportion of the VO₂(M1) phase in the mixed phase of the VO₂(M1) phase and the other vanadium oxide can be adjusted by adjusting a temperature at which the chemical reaction is performed in the subcritical fluid and/or the supercritical fluid. As illustrated in FIG. 2, as the temperature at which the chemical reaction is performed in the subcritical fluid and/or the supercritical fluid is increased, a transition of the other vanadium oxide, V₆O₁₃, to the VO₂(M1) phase is promoted.

The chemical reaction in the subcritical fluid and/or the supercritical fluid is preferably a chemical reaction in the supercritical fluid.

In addition to the above, necessary treatments such as purification treatment can be appropriately performed.

### Diaper

A diaper according to the invention includes the semiconductor type gas sensor according to the invention.

The diaper other than the semiconductor type gas sensor according to the invention is not particularly limited, and a commercially available product can be used, and the method for fixing to the diaper is also not particularly limited as long as urine and/or feces can be detected, and can be performed by a normal fixing method.

The diaper according to the invention can detect urine and/or feces regardless of whether the semiconductor type gas sensor according to the invention is provided inside (on a body side) or outside the diaper.

### Method for Detecting Gas

The method for detecting gases according to the invention is a method for distinguishing and detecting the ammonia gas and the hydrogen sulfide gas or the like using the semiconductor type gas sensor according to the invention.

Environmental conditions for detecting gases (temperature, concentration of gas to be detected, contact with gas to be detected, and the like) are as described in the above-described semiconductor type gas sensor according to the invention.

### Method for Detecting Urine and/or Feces

As described above, the semiconductor type gas sensor according to the invention can be used to detect urine and/or feces by distinguishing and detecting urine and feces. That is, according to the invention, the ammonia gas and the hydrogen sulfide gas can be distinguished and detected by the semiconductor type gas sensor according to the invention, and presence or absence of urine excretion can be distinguished and detected using the ammonia gas and presence or absence of feces excretion can be distinguished and detected using the hydrogen sulfide gas.

### Method for Determining Diaper Replacement Time

The method for determining a diaper replacement time according to the invention includes determining the diaper replacement time by detecting the ammonia gas derived from urine and/or the hydrogen sulfide gas derived from feces excreted on the diaper using the semiconductor type gas sensor according to the invention.

The diaper replacement time can be freely determined by setting the diaper replacement time so that an alarm is sounded according to a gas concentration detected by the semiconductor type gas sensor.

For example, regarding the diaper replacement time due to excretion of urine, one method is to notify the replacement time by an alarm or the like when a response in which the response ΔR/R (Response (%)) represented by formula (A) to be described later exceeds a predetermined threshold due to contact with the ammonia gas exceeds a predetermined number of times. With this method, the diaper can be replaced at a timing not exceeding a urine tolerance of the diaper. Regarding the diaper replacement time due to excretion of feces, one method is to notify the replacement time by an alarm or the like when the response ΔR/R (Response (%)) represented by formula (A) to be described later falls below a predetermined threshold due to contact with the hydrogen sulfide gas so that the diaper can be replaced after one bowel movement. By this method, the diaper can be replaced at the timing of defecation. As illustrated in Example 3 to be described later, even when the excretion of urine and the excretion of feces are performed at approximately the same time, one method is to notify the replacement time by an alarm or the like when there is a significant difference such that an absolute value of a change in a negative direction of the response ΔR/R (Response (%)) represented by formula (A) to be described later is, for example, twice or more an absolute value of a change in a positive direction of the response ΔR/R (Response (%)) represented by formula (A) to be described later generated immediately before the change in the negative direction. By this method, the diaper can be replaced at the timing of defecation.

Hereinafter, regarding an example of excellent characteristics of the VO₂(M1) phase as the gas-sensing material, test results of the semiconductor type gas sensor using the VO₂(M1) phase single phase and the mixed phase of the VO₂(M1) phase and the V₆O₁₃ will be shown below as an example. The invention is not to be construed as being limited by the following examples except for being specified in the invention. min means a minute.

### EXAMPLES

### Synthesis Example 1: Synthesis of VO₂(M1) Phase Powder

(1) Preparation of Precursor In a container, 5 mL of an H₂O₂ aqueous solution (30%)

and V₂O₅ (0.05 mol) were added and stirred at 60° C. for 24 hours, and then N₂H₄·H₂O (2.75 mmol) was further added and stirred at room temperature (25° C.) for 20 minutes to obtain a gel precursor.

### (2) Synthesis of VO₂(M1) Phase Powder

The obtained gel precursor (5 g) was dispersed in 10 mL of water, a chemical reaction in a subcritical fluid or a supercritical fluid (subcritical water or supercritical water) was performed for 30 minutes to 60 minutes, suction filtration was performed, and vacuum drying was performed at 60° C. to obtain a single phase VO₂(M1) powder or a powder of a mixed phase of the VO₂(M1) phase and V₆O₁₃.

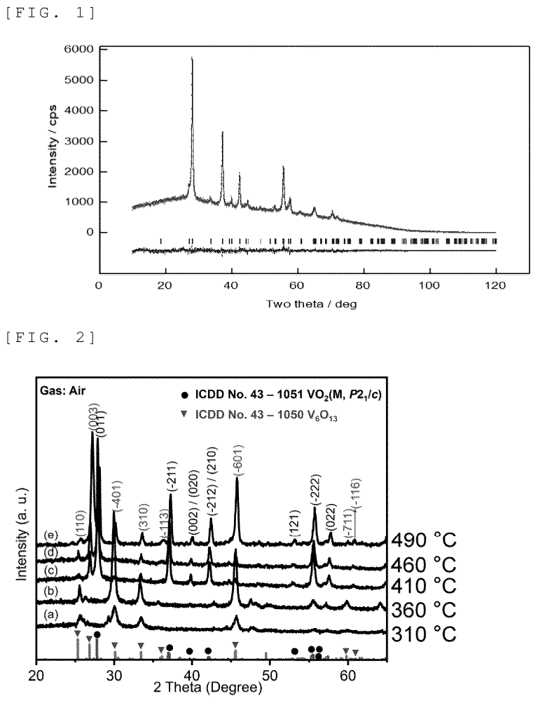

The single phase VO₂(M1) powder was obtained by performing a step of preparation of precursor in the above (1) in an atmosphere replaced with a nitrogen gas, and performing the chemical reaction in the supercritical fluid (supercritical water) in the above (2) at 490° C. An XRD pattern of the obtained powder is illustrated in FIG. 1. In FIG. 1, an upper pattern is the XRD pattern of the single phase VO₂(M1)powder, and a lower pattern is an XRD pattern theoretically calculated based on a crystal structure model with respect to a measured diffraction pattern by Rietveld analysis.

The powder of the mixed phase of the VO₂(M1) phase and the V₆O₁₃ was obtained by performing the step of preparation of precursor in the above (1) in the atmosphere and performing the chemical reaction in the supercritical fluid (supercritical water) in the above (2) at 410° C., 460° C., or 490° C. Meanwhile, when the step of preparation of precursor in the above (1) was performed in the atmosphere, and the chemical reaction in the subcritical fluid (subcritical water) in the above (2) was performed at 310° C. or 360° C., the VO₂(M1) phase was hardly obtained, and a powder mainly containing the V₆O₁₃ was obtained. An XRD pattern of the obtained powder is illustrated in FIG. 2. In FIG. 2, (a) to (e) of FIG. 2 are XRD patterns of powders obtained by performing chemical reactions in a subcritical fluid or a supercritical fluid (subcritical water or supercritical water) at 310° C., 360° C., 410° C., 460° C., and 490° C., respectively, and (c) to (e) of FIG. 2 are XRD patterns of powders of a mixed phase of a VO₂(M1) phase and V₆O₁₃. At a bottom of (a) to (e) of FIG. 2, the XRD pattern of the VO₂(M1) phase (ICDD No. 43-1051 VO₂(M1)) and the XRD pattern of the V₆O₁₃ (ICDD No. 43-1050 V₆O₁₃) registered in International Centre for Diffraction Data (ICDD) are superimposed. A characteristic peak of the VO₂(M1) phase is indicated by •, and a characteristic peak of the V₆O₁₃ is indicated by ▾.

### Identification

The obtained powder was identified by analysis of the XRD pattern using an X-ray diffractometer (XRD, manufactured by Bruker, D2 Phaser).

The XRD pattern illustrated in FIG. 1 illustrated characteristic peaks in an XRD pattern of the VO₂(M1) phase registered in International Centre for Diffraction Data (ICDD) (ICDD No. 43-1051 VO₂(M1), illustrated in (a) in FIG. 3), which matched well, confirming that a VO₂(M1) phase powder was obtained. Also, (b) in FIG. 3 illustrates an XRD pattern of a VO₂(B) phase (ICDD No. 81-2392 VO₂(B)) registered in ICDD.

In the XRD pattern in FIG. 2, peaks illustrated as (011), (−211), (002)/(020), (−212)/(210), (121), (−222), and (022) in order from a 2θ =0°side are respectively attributed to peaks characteristic of the VO₂(M1) phase (peaks indicated by • in the drawing), and peaks illustrated as (110), (003), (−401), (310), (−113), (−601), (−711), and (−116) in order from the 2θ=0°side are respectively attributed to peaks characteristic of the V₆O₁₃ (peaks indicated by ▾ in the drawing). Based on these results, it was confirmed that the powder obtained by performing the step of preparation of precursor in the above (1) in the atmosphere and performing the chemical reaction in the supercritical fluid (supercritical water) in the above (2) at 410° C., 460° C., or 490° C. was a powder of a mixed phase of the VO₂(M1) phase and the V₆O₁₃.

A composition (molar ratio) of the powder of the mixed phase of the VO₂(M1) phase and the V₆O₁₃ was analyzed by powder X-ray diffraction (XRD) to be VO₂(M1) phase: V₆O₁₃=0.70:1 (that is, about 41:59), under the condition of 410° C., VO₂(M1) phase: V₆O₁₃=0.99:1 (that is, about 50:50) under the condition of 460° C., and VO₂(M1) phase: V₆O₁₃=1.06:1 (that is, about 51:49) under the condition of 490° C., and was calculated and confirmed by a ratio of peak areas having the strongest intensity of each phase.

### Synthesis Example 2: Production of Gas Detection Device

### (1) Production of Interdigitated Electrode Sheet

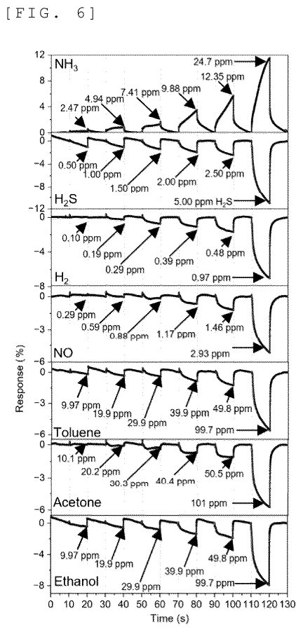

On a hydroxypropyl methylcellulose (HPMC) film having a thickness of 33 μm, a silver paste (product name: BASE-CD01, manufactured by Shanghai Mifang Electronic Technology) was applied under conditions of 1 mm/s and 50 kPa using an electrode production facility (product name: Scientific 3, manufactured by Shanghai Zhongbin Technology) to produce a flexible interdigitated electrode sheet (see FIGS. 4A and 4B: FIG. 4A is a photograph of the interdigitated electrode sheet bent along a longitudinal direction, and FIG. 4B is a photograph of the interdigitated electrode sheet bent perpendicularly to the longitudinal direction). An interdigitated portion (portion not including a lead wire) of an interdigitated electrode had a length of 15 mm ×a width of 5 mm, an electrode spacing of 0.50 mm, and an electrode width of 0.50 mm.

### (2) Production of Semiconductor Type Gas Sensor

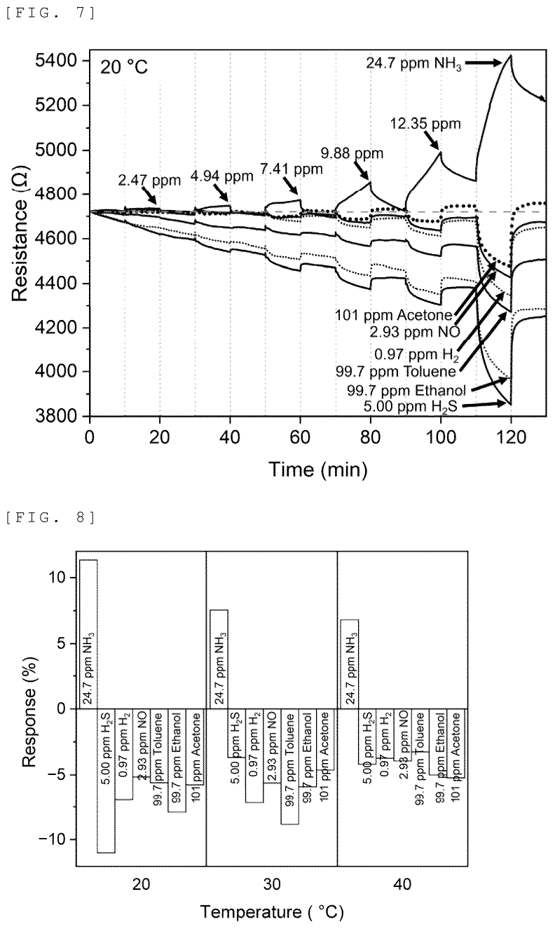

In a container containing 200 μL of ethanol, 0.010 g of the powder of the VO₂(M1) phase single phase or the powder of the mixed phase of the VO₂(M1) phase and the V₆O₁₃ obtained in Synthesis Example 1 was added and dispersed by ultrasonic waves. An obtained slurry (15 μL) was dropped onto the interdigitated electrode of the interdigitated electrode sheet using a pipette. After the slurry was spread over the electrode, another 15 μL was dropped, and the slurry was dropped four times in total, and then the dropped slurry was dried in a drying oven at 60° C. overnight to produce a semiconductor type gas sensor chip in which a thickness of a sensing layer (that is, a VO₂(M1) phase single phase layer or a layer of the mixed phase of the VO₂(M1) phase and the V₆O₁₃) was about 1.8 μm (see FIG. 5).

The obtained semiconductor type gas sensor chip was fixed to a chamber for a gas-sensing device.

Example 1: Evaluation of Gas Response of VO₂(M1) Phase

### (1) Semiconductor Type Gas Sensor Chip Using VO₂(M1) Phase Single Phase Layer

The gas response of the semiconductor type gas sensor chip using the VO₂(M1) phase single phase layer as the sensing layer produced above was collected using a data collection device (Agilent 34970A, manufactured by Agilent Technologies) with a two-point probe method. Various analysis target gases containing NH3, H2, H₂S, NO, toluene, ethanol, and acetone at a predetermined concentration were introduced into a sensor device for each gas, and a gas response of the semiconductor type gas sensor in a dry nitrogen base at room temperature of (20° C.) was measured.

For the nitrogen-based gas, the target gas at each concentration was mixed with a nitrogen gas and flowed at a total flow rate of 200 standard cubic centimeter per min (SCCM). A flow rate of the 200 SCCM target gas corresponds to the concentration. An injection time of an analyte was 10 minutes, with an interval of 10 minutes to reintroduce a nitrogen atmosphere. The response ΔR/R (Response (%)) was calculated using the following formula (A) as a ratio of an electrical resistance value (R0) of a sensor in nitrogen immediately before a flow of the target gas at each concentration to an electrical resistance value (R_{gas}) of a sensor in an analysis gas.

ΔR/R={(R_{gas}−R₀)/R₀}×100% formula (A)

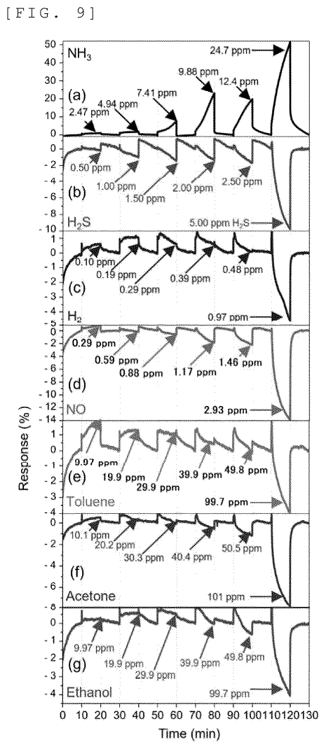

Results of the obtained gas response are illustrated in FIGS. 6 to 8. In FIG. 6, a vertical axis represents a response ΔR/R (Response (%)). The gas responses to the gas types at each concentration illustrated in the drawing with respect to each of the gas types, an NH₃ (ammonia) gas, an H₂S (hydrogen sulfide) gas, an H₂ (hydrogen) gas, an NO (nitrogen monoxide) gas, a toluene gas, an acetone gas, and an ethanol gas, are illustrated in order from a top. In FIG. 7, a vertical axis represents an electrical resistance value (R_{gas}) (Resistance (Ω)), and a gas concentration of each gas type is the same as the concentration illustrated in FIG. 6. In FIG. 8, the gas response at 20° C., the gas response at 30° C., and the gas response at 40° C. are illustrated in order from a left for each region separated by a straight line, and seven bar graphs in each region illustrate the gas response to each of the gas types, 24.7 ppm NH₃ gas, 5.00 ppm H₂S gas, 0.97 ppm H₂ gas, 2.93 ppm NO gas, 99.7 ppm toluene gas, 99.7 ppm ethanol gas, and 101 ppm acetone gas, in order from the left.

As illustrated in FIGS. 6 and 7, after exposing the semiconductor type gas sensor to the target gas for 10 minutes, a response value at 20° C. illustrated a positive behavior with an increase in electrical resistance with respect to an NH₃ gas having a gas concentration of 2.47 ppm to 24.7 ppm, and a negative behavior with a decrease in electrical resistance with respect to any of 0.50 ppm to 5.00 ppm H₂S gas, 0.10 ppm to 0.97 ppm H₂ gas, 0.29 ppm to 2.93 ppm NO gas, 9.97 ppm to 99.7 ppm toluene gas, 9.97 ppm to 99.7 ppm ethanol gas, and 10.1 ppm to 101 ppm acetone gas. As illustrated in FIG. 8, at 20° C., 30° C., and 40° C., a positive behavior was always illustrated with respect to the NH₃ gas, and a negative behavior was always illustrated with respect to the H₂S gas, the H₂ gas, the NO gas, the toluene gas, the ethanol gas, and the acetone gas.

(2) Semiconductor Type Gas Sensor Chip Using Layer of Mixed Phase of VO₂(M1) Phase and V₆O₁₃

A gas response of the semiconductor type gas sensor chip using the layer of the mixed phase of the VO₂(M1) phase and the V₆O₁₃ produced above instead of the semiconductor type gas sensor chip using the VO₂(M1) phase single phase layer in the above (1) was measured and evaluated in the same manner as in (1).

As the layer of the mixed phase of the VO₂(M1) phase and the V₆O₁₃, a layer obtained using a powder of the mixed phase of the VO₂(M1) phase and the V₆O₁₃ obtained by performing the step of preparation of precursor in the above (1) in the atmosphere and performing the chemical reaction in the supercritical fluid (supercritical water) at 490° C. for 30 minutes in the above (2) in Synthesis Example 1 was used.

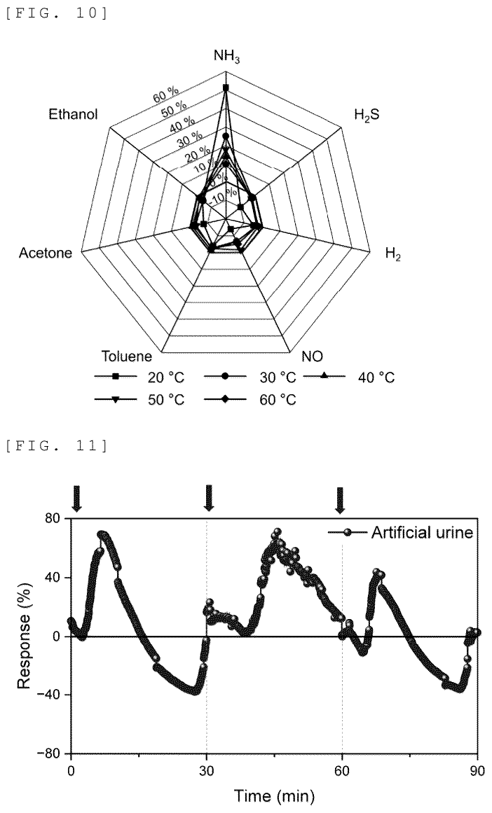

Results of the obtained gas response are illustrated in FIGS. 9 and 10. In FIG. 9, a vertical axis represents the response ΔR/R (Response (%)), and (a) to (g) in FIG. 9 respectively illustrate gas responses to the gas types at each concentration illustrated in the figure for each gas type, the NH₃ gas, the H₂S gas, the H₂ gas, the NO gas, the toluene gas, the acetone gas, and the ethanol gas, in order from a top. In FIG. 10, the responses ΔR/R (Response (%)) to the gas types, 24.7 ppm NH3 gas, 5.00 ppm H₂S gas, 0.97 ppm H₂ gas, 2.93 ppm NO gas, 99.7 ppm toluene gas, 101 ppm acetone gas, and 99.7 ppm ethanol gas, at 20° C. (▪), 30° C. (•), 40° C. (▴), 50° C. (▾), and 60° C. (♦) are illustrated, respectively.

As illustrated in (a) to (g) in FIG. 9, a response value after exposing the semiconductor type gas sensor to the target gas for 10 minutes illustrated a positive behavior with an increase in electrical resistance with respect to the NH₃ gas having a gas concentration of 2.47 ppm to 24.7 ppm at 20° C., and illustrated a negative behavior with a decrease in electrical resistance with respect to any of 0.50 ppm to 5.00 ppm H₂S gas, 0.10 ppm to 0.97 ppm H₂ gas, 0.29 ppm to 2.93 ppm NO gas, 9.97 ppm to 99.7 ppm toluene gas, 10.1 ppm to 101 ppm acetone gas, and 9.97 ppm to 99.7 ppm ethanol gas. As illustrated in FIG. 10, after exposing the semiconductor type gas sensor to the target gas for 10 minutes, response values at 20° C., 30° C., 40° C., 50° C., and 60° C. always illustrated a positive behavior (response ΔR/R (Response (%)) exceeded 0%) with an increase in electrical resistance with respect to NH₃ gas having a gas concentration of 24.7 ppm, and always illustrated a negative behavior (response ΔR/R (Response (%)) was less than 0%) with a decrease in electrical resistance with respect to any of 5.00 ppm H₂S gas, 0.97 ppm H2 gas, 2.93 ppm NO gas, 99.7 ppm toluene gas, 101 ppm acetone gas, and 99.7 ppm ethanol gas.

### Example 2: Evaluation of Diaper Sensor (Inside Mode)

A diaper (manufactured by Unicharm, product name: Trepanman L size) was attached to a doll having a waist circumference of 51 cm, the semiconductor type gas sensor chip used in Example 1 (2) was fixed to the diaper using a double-sided tape at a position inside the diaper, at 135° (position corresponding to one side of buttocks of the doll) when a navel was set to 0° around the waist of the doll, with a sensing layer facing the doll and not being covered by a fixing tape, and a response to artificial urine (composition: 97.15% by mass of water, 1.75% by mass of urea, 0.55% by mass of NaCl, and 0.55% by mass of KCl) and a response to the H₂S gas (5 ppm H₂S gas mimicking a volatile gas derived from feces) were evaluated at 25° C.

The response to the artificial urine was evaluated by injecting to supply 40 mL, 38° C. artificial urine every 30 minutes into a center position of a front surface of the diaper (a navel side of the doll) inside the diaper using a syringe. The response to the H₂S gas (5 ppm H₂S gas mimicking the volatile gas derived from feces) was evaluated by continuously supplying 5 ppm H₂S gas for 60 seconds at each timing of the start of measurement (0 min), 30 min later, and 60 min later to the inside of the diaper at a center position at a rear of the diaper (on a rear of the doll) through a silicon tube having an inner diameter of 4 mm.

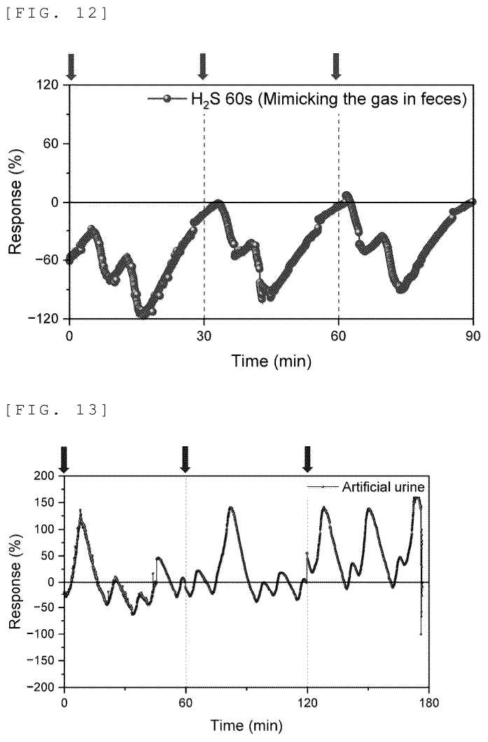

FIG. 11 illustrates a result of the response to the artificial urine, and FIG. 12 illustrates a result of the response to the H₂S gas. In FIG. 11, a vertical axis represents the response ΔR/R (Response (%)), and a timing of supplying the artificial urine is indicated by an arrow. In FIG. 12, a vertical axis represents the response ΔR/R (Response (%)), and a timing of supplying the H₂S gas is indicated by an arrow.

As illustrated in FIG. 11, when the artificial urine was supplied, a first supply (0 min) and a second supply (30 min) each illustrated a positive behavior with the response ΔR/R of about 70%, and a third supply (60 min) illustrated a positive behavior with the response ΔR/R of about 40%. As illustrated in FIG. 12, when the H₂S gas was supplied, the response ΔR/R illustrated a negative behavior of about −120% in a first supply (0 min), the response ΔR/R illustrated a negative behavior of about −100% in a second supply (30 min), and the response ΔR/R illustrated a negative behavior of about-90% in a third supply (60 min). Since the artificial urine and the H₂S gas were supplied under a normal pressure environment, the response ΔR/R was not necessarily 0% at a supply timing of the artificial urine or the H₂S gas due to diffusion of various gases, but a clear negative or positive behavior was illustrated as described above.

As described above, it can be seen that urine and feces can be distinguished and detected by using the semiconductor type gas sensor according to the invention as the diaper sensor (inside mode).

### Example 3: Evaluation of Diaper Sensor (Outside Mode)

In Example 2 described above, the semiconductor type gas sensor chip used in Example 1 (2) was fixed to the outside of the diaper at 90°(position corresponding to a waist side surface of the doll) when a portion corresponding to the navel around the waist of the diaper was set to 0°using a double-sided tape with the sensing layer facing the doll and not being covered by a fixing tape. In the Example 2, the response to the artificial urine and the response to the H₂S gas (5 ppm H₂S gas mimicking a volatile gas derived from feces) were evaluated at 25° C. in the same manner as in Example 2, except that the semiconductor type gas sensor chip was fixed to the outside of the diaper as described above, and an evaluation of the response to the H₂S gas was changed as follows. Regarding the response to the H₂S gas, in any of the first supply of the H₂S gas (0 min), the second supply of the H₂S gas (30 min), and the third supply of the H₂S gas (60 min), simultaneously with the supply of the H₂S gas, 40 mL, 38° C. artificial urine was supplied by injecting the artificial urine into the center position of the front surface of the diaper (the navel side of the doll) inside the diaper using a syringe, and the response when the H₂S gas and the artificial urine were simultaneously supplied was evaluated.

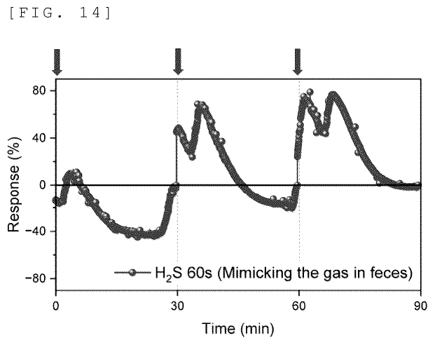

FIG. 13 illustrates a result of the response to the artificial urine, and FIG. 14 illustrates a result of the response to a simultaneous supply of the H₂S gas and the artificial urine. In FIG. 13, a vertical axis represents the response ΔR/R (Response (%)), and a timing of supplying the artificial urine is indicated by an arrow. In FIG. 14, a vertical axis represents the response ΔR/R (Response (%)), and a timing of supplying the H₂S gas and the artificial urine is indicated by an arrow.

As illustrated in FIG. 13, when the artificial urine was supplied, a first supply (0 min) and a second supply (60 min) each illustrated a positive behavior with the response ΔR/R of about 130%, and in a third supply (120 min), a positive behavior (p-type behavior) with the response ΔR/R of about 130% was illustrated three times within 60 minutes after the supply. As illustrated in FIG. 14, a negative behavior (n-type behavior) was illustrated in any case of the first supply (0 min), the second supply (30 min), and the third supply (60 min) in which the H₂S gas and the artificial urine were simultaneously supplied. As in Example 2, also in Example 3, since the artificial urine and the H₂S gas were supplied under a normal pressure environment, the response ΔR/R was not necessarily 0% at a supply timing of the artificial urine and/or the H₂S gas due to diffusion of various gases, but a clear negative or positive behavior was illustrated as described above.

As described above, it can be seen that urine and feces can be distinguished and detected by using the semiconductor type gas sensor according to the invention as the diaper sensor (outside mode).

What is claimed is:

1. A semiconductor type gas sensor comprising:
an electrode; and
a VO₂(M1) phase containing layer provided on the electrode, wherein
a VO₂(M1) phase functions as a gas-sensing material.

2. The semiconductor type gas sensor according to claim 1, wherein
the electrode and the VO₂(M1) phase containing layer are provided on/above a substrate in this order.

3. The semiconductor type gas sensor according to claim 1, which is for use in distinguishing and detecting an ammonia gas and at least one gas among hydrogen sulfide, hydrogen, nitrogen monoxide, toluene, acetone, and ethanol.

4. The semiconductor type gas sensor according to claim 1, which is for use at an operation temperature of 15° C. to 65° C.

5. The semiconductor type gas sensor according to claim 1, which has an increase in electrical resistance when exposed to an ammonia gas, and has a decrease in electrical resistance when exposed to at least one gas of hydrogen sulfide, hydrogen, nitrogen monoxide, toluene, acetone, and ethanol.

6. The semiconductor type gas sensor according to claim 2, wherein
the substrate is a flexible substrate.

7. The semiconductor type gas sensor according to claim 5, which is for use in detection of urine and/or feces.

8. The semiconductor type gas sensor according to claim 7, which is for use by being attached to a diaper.

9. A diaper comprising:
the semiconductor type gas sensor according to claim 8.

10. A method for detecting gases, comprising:
distinguishing and detecting an ammonia gas and at least one gas among hydrogen sulfide, hydrogen, nitrogen monoxide, toluene, acetone, and ethanol using the semiconductor type gas sensor according to claim 1.

11. A method for detecting urine and/or feces, comprising:
distinguishing and detecting urine and feces using the semiconductor type gas sensor according to claim 1.

12. A method for determining a diaper replacement time, comprising:
determining a diaper replacement time by detecting an ammonia gas derived from urine and/or a hydrogen sulfide gas derived from feces excreted on a diaper using the semiconductor type gas sensor according to claim 7.

13. A gas-sensing material comprising:
a VO₂(M1) phase as an active component.

14. The gas-sensing material according to claim 13, which is for use in distinguishing and detecting an ammonia gas and at least one gas among hydrogen sulfide, hydrogen, nitrogen monoxide, toluene, acetone, and ethanol.

15. The gas-sensing material according to claim 14, which is for use in detection of urine and/or feces.
